(19) United States
(12) Patent Application Publication
(10) Pub. No.: US 2008/0221678 A1
(43) Pub. Date: Sep. 11, 2008

(54) Collapse-Resistant Breast Implant With Partial Internal Shells

(75) Inventor: Robert S. Hamas, Dallas, TX (US)

Correspondence Address: THE WEBB LAW FIRM, P.C., 700 KOPPERS BUILDING, 436 SEVENTH AVENUE, PITTSBURGH, PA, 15219, US

(73) Assignee: IDEAL IMPLANT INCORPORATED, Dallas, TX (US)

(21) Appl. No.: 12/043,507

(22) Filed: Mar. 6, 2008

Related U.S. Application Data
(60) Provisional application No. 60/905,232, filed on Mar. 6, 2007.

(51) Int. Cl. A61F 2/12

(52) U.S. Cl. 623/8

(57) ABSTRACT

A surgically-implantable breast prosthesis comprises a first shell enclosing a lumen; a second shell enclosing a lumen, and one or more fitted partial shells situated between the exterior of the second shell and the interior of the first shell.

### BACKGROUND OF THE INVENTION

1. Field of the Invention

This invention relates generally to surgically-implantable mammary prostheses.

2. Description of Related Art

Breast prostheses have long been used for breast augmentation and for reconstructive surgery following a mastectomy. The prostheses are available in numerous sizes and shapes including teardrop, round, low profile, and high profile. Usually, breast prostheses are implanted via a small inframammary or peri-aerolar incision into a pocket dissected deep into the patient's own breast tissue in front of the pectoral muscle. In certain situations, the prosthesis may be placed behind the various chest muscles.

U.S. Pat. No. 5,480,430 entitled “Shape-Retaining Shell for a Fluid Filled Prosthesis” discloses a prosthesis shell that asymmetrically varies in thickness and, in particular, the wall of the upper portion of the shell is thickened so as to prevent collapsing of the shell near the uppermost edge. In the examples disclosed in that patent, the prosthesis itself has an asymmetric outer configuration and the proper orientation at the time of implanting is readily apparent. Many breast prostheses have a symmetrical outer configuration. It would require extra precautions to recognize the portion of the prosthesis having the thicker wall that must be oriented in the superior position at the time of implanting. Moreover, rotation of the prosthesis after implanting is likely unless prevented by various surgical means. Furthermore, since the edge is thickened, it would be more palpable through the skin, especially of a thin patient.

There are breast prosthetic devices utilizing an outer envelope wherein the envelope contains baffle-forming material. The baffle-forming material fills at least a portion of the outer envelope, while the remainder of the outer envelope is filed with a fluid, such as saline solution. The baffle-forming material may or may not be attached to the outer envelope. The drawback to such prior art baffle-forming material is that such material does not match the single layer structure, geometry, proportions, etc., of the outer envelope, thereby resulting in wrinkling and folding of the implant due to the uncontrolled position of the baffle-forming material. Additionally, some of the prior art baffle material can be felt through the implant, resulting in an unnatural feel to the implant.

In my U.S. Pat. No. 6,802,861 entitled “Structured Breast Implant,” I disclose and claim a breast implant having a plurality of internal fitted shells. This implant overcame some of the drawbacks of the prior art implants. This application is directed to an improvement over my earlier invention. It is an advantage, according to the present invention, to provide an implantable breast prosthesis that has improved resistance to collapsing near the top and sagging when the patient and implant are upright and also having a softer edge that is less likely felt through the skin of a thin patient.

### SUMMARY OF THE INVENTION

Briefly, according to the present invention, there is provided a surgically-implantable prosthetic device comprising a shell enclosing a lumen able to accommodate a fluid therein. The exterior surface of the outer shell defines an edge portion of the shell as viewed from a front direction. The edge portion separates front and rear portions of the outer shell. The prosthetic device further comprises an inner shell having an exterior surface, an interior surface, and enclosing an inner lumen or cavity, wherein the inner lumen is able to accommodate a second fluid therein. Additionally, the prosthetic device has one or more fitted partial shells situated between the exterior surface of the inner shell and the interior surface of the outer shell. The fitted partial shells are positioned adjacent to the inner surface of the outer shell. The fitted partial shells have a generally circular or teardrop shape and a diameter so as to be spaced from the edge portion. It is an improvement that the fitted partial shells have the effect of stiffening central front and/or rear surfaces of the outer shell, yet spaced from the edge portion of the outer shell so the edge is soft to palpation.

Both the outer lumen of the outer shell and the inner lumen of the inner shell may be filled with a fluid. The fluid is able to move within the outer lumen and envelop the fitted partial shells. A saline solution would be an appropriate choice for use as the fluid. Saline refers to any electrolyte combination together with water, however, the invention is not limited solely to the use of saline. Other fluids may be utilized, such as, for example, organic polymers or protein fluids; furthermore, certain gases may possibly be utilized as substitutes for fluids. Lubricating agents may be added to the saline. The outer lumen and/or the inner lumen may be pre-filled prior to implantation or, alternatively, may be first implanted and then filled with the fluid. One or more valves may be provided for the filling of the outer lumen, which includes the spaces between the fitted shells or the inner lumen.

The advantages of the present invention will be understood from the description of the desirable embodiments, taken with the accompanying drawings, wherein like reference numerals represent like elements throughout.

### BRIEF DESCRIPTION OF THE DRAWINGS

FIG. 1 is a schematic view of an implant, according to the present invention, having multiple fitted partial shells; and

FIG. 2 is a front view of an implant, according to the present invention.

### DESCRIPTION OF THE PREFERRED EMBODIMENTS

For purposes of the description hereinafter, the spatial or directional terms, such as “inner,” “outer,” “top,” “bottom,” “central,” and derivatives thereof, shall relate to the present invention as it is oriented in the drawing figures. The “rear portion” of a breast implant is that intended to be adjacent the chest wall and the “front portion” is that portion facing away from the chest wall. However, it is to be understood that the present invention may assume various alternative variations, except where expressly specified to the contrary. It is also to be understood that the specific apparatus illustrated in the attached drawings and described in the following specification is simply an exemplary embodiment of the present invention. Hence, specific dimensions and other physical characteristics related to the embodiments disclosed herein are not to be considered as limiting.

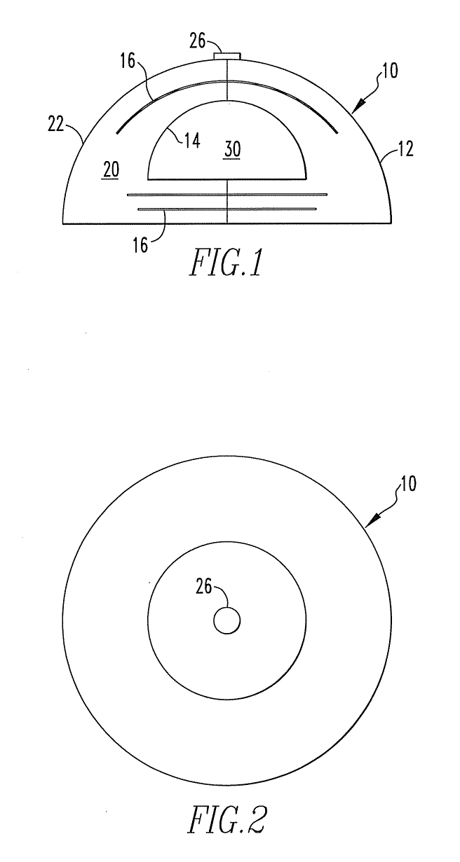

Implant 10 is illustrated in FIG. 1. The implant 10 is particularly adapted for use as a surgically-implantable mammary prosthesis. The implant 10 includes an outer shell 12 enclosing a lumen or a cavity, an inner shell 14 enclosing a lumen or a cavity, and one or more generally dome shaped in front, flat in rear, fitted partial shells 16. In a particularly desirable embodiment, with reference to the implant 10 as it is oriented in the drawings, the dimensions of the outer shell 12 and the inner shell 14 are defined by a diameter measurement and a projection measurement. The diameter measurement is representative of the width of the implant 10 at its widest point and the projection measurement is representative of the height of the implant 10 at its tallest point. In this desirable embodiment, the diameter measurement of the implant 10 is greater than the projection measurement of the implant 10.

With reference to FIG. 1, the outer shell 12 defines an outer lumen 20 and includes an exterior surface and an interior surface. The outer shell 12 may include a valve 26 that bridges a portion between an exterior surface 22 and the interior surface of the outer shell 12. The valve 26 may be placed along various areas of the outer shell 12. The valve 26 allows for filling of the outer lumen 20 of the outer shell 12 with a fluid after the manufacture of the implant 10, either before or after implantation into a patient. The fluid is preferably a saline solution, yet it is to be understood that the term fluid may refer to both gaseous and liquid fillers or any combination thereof including, but not limited to, electrolyte and organic solutions. The valve 26 also allows for the controlled removal of the fluid without damaging or destroying the implant 10. Alternatively, the outer lumen 20 may be manufactured as a pre-filled and completely sealed member (not shown), and therefore, not require a valve 26 for the outer lumen 20.

The outer shell 12 is preferably constructed of a non-porous, flexible, biocompatible material, such as silicone elastomer. The outer shell 12 has a wall of sufficient thickness to provide structural integrity to retain fluids while achieving the desired flexibility and malleability of the implant 10. The outer shell 12 is substantially oval-shaped as viewed from the side and a generally circular or teardrop shape as viewed from the front. Thus, the shape of the implant 10 is defined by the overall external shape of the outer shell 12. In an exemplary embodiment, the enclosed volume within the outer shell 12 is 375 cc. Therefore, the outer shell 12 may accommodate say 375 cc of volume-displacing material, e.g., fluid and fitted partial shells. It is to be understood that various other volumes of shells 12 may be utilized.

The inner shell 14 defines an inner lumen 30, and includes an exterior surface and an interior surface. The inner shell 14 is smaller than the outer shell 12 in that the diameter measurement and/or the projection measurement is less than that of the implant 10. In an exemplary embodiment, the enclosed volume of the inner shell 14 is 250 cc. The inner shell 14 is situated within the outer lumen 20 of the outer shell 12 in a relatively central position with respect to the interior surface of the outer lumen 20. Similar to the outer shell 12, the inner shell 14 may include a valve. The valve bridges the exterior surface and the interior surface of the inner shell 14, as well as the exterior surface and the interior surface of the outer shell 12. The valve (not shown) allows for filling of the inner lumen 30 of the inner shell 14 with the fluid after the manufacture of the implant 10, either before or after implantation into a patient. The valve (not shown) also allows for the controlled removal of fluid without damaging or destroying the implant 10. Alternatively, the inner lumen 30 may be manufactured as a pre-filled and completely sealed member and, therefore, not requiring the valve.

Returning to FIG. 1, once implanted, the top or front of the implant 10 faces away from the chest wall of a patient. The valves may be situated along any area of the exterior surface of the outer shell 12.

One or more fitted partial shells are situated within the outer lumen 20 of the outer shell 12. It is to be appreciated that there exists an optimal number of fitted partial shells for effectively achieving the objects of the present invention. The optimal number of fitted partial shells, which would be apparent to one having ordinary skill in the art, is based upon the characteristics of the implant, e.g., the needs of the patient, the dimensions of the implant, the type of fluid used, etc. Each fitted partial shell may be formed from a flexible, biocompatible material, such as silicone elastomer, having similar construction in shape as that of the inner shell 14 or the outer shell 12. Desirably, the fitted shells are to be as thin as possible, so as to minimize any bulk within the implant 10.

The fitted partial shells are positioned to be adjacent the inner surface of the front face of the outer shell and/or adjacent the back face of the outer shell. The fitted partial shells have a generally circular or teardrop shape and are of a diameter so as to be spaced from the edge portions of the implant. The fitted partial shells may be tethered so as to remain positively centered on the front or back face of the implant. The fitted partial shells may simply be held centered due their domed configuration and friction. The fitted partial shells can slide relative to the inner surface of the outer shell and relative to each other. However, there appears to be an attraction between the surfaces of the fitted partial shells and the inner surface of the outer shell and/or the outer surface of the inner shell. In some manner not entirely understood, this structural arrangement has the effect of stiffening the central portions of the front and/or back of the outer shell. This, in turn, has the effect of reducing the tendency of the upper portion to collapse when the implant is upright while at the same time not thickening the edge portion so it is not felt through the skin.

Having thus described my invention with the detail and particularity required by the Patent Laws, what is desired protected by Letters Patent is set forth in the following claims.

The invention claimed is:

1. A surgically-implantable breast prosthesis comprising: a first enclosing shell having an exterior surface, an interior surface, and enclosing a lumen, said exterior surface defining an edge portion of said first enclosing shell as viewed from a front direction, said edge portion separating front and rear portions of said first enclosing shell, wherein the lumen enclosed by the first shell is able to accommodate a first fluid therein; a second enclosing shell having an exterior surface, an interior surface, and enclosing a lumen, wherein the lumen enclosed by the second shell is able to accommodate a second fluid therein; and one or more fitted partial shells situated between the exterior surface of the second shell and the interior surface of the first shell such that all surfaces of the fitted partial shells are in communication with the first fluid, the fitted partial shells being positioned adjacent the front and/or back face of the first enclosing shell.

2. A surgically-implantable breast prosthesis according to claim 1, wherein the edge portion of the first enclosing shell is generally circular.

3. A surgically-implantable breast prosthesis according to claim 1, wherein the one or more fitted partial shells are adjacent to each other.

4. A surgically-implantable breast prosthesis according to claim 1, wherein the one or more fitted partial shells comprise an innermost shell and an outermost shell, wherein the innermost shell is adjacent to the exterior surface of the second shell and the outermost shell is adjacent to the interior surface of the first shell.

5. A surgically-implantable breast prosthesis according to claim 1, wherein each fitted partial shell is generally dome shaped.

6. A surgically-implantable breast prosthesis according to claim 1, wherein the distance between each of the one or more fitted partial shells is between 0 cm and 1.0 cm.
